(19) United States
(12) Patent Application Publication
(10) Pub. No.: US 2008/0195000 A1
(43) Pub. Date: Aug. 14, 2008

(54) System and Method for Dermatological Treatment Using Ultrasound

(76) Inventors: Gregory J.R. Spooner, Kensington, CA (US); Scott A. Davenport, Half Moon Bay, CA (US); Steven Christensen, Fremont, CA (US); David A. Gollnick, San Francisco, CA (US)

Correspondence Address: STALLMAN & POLLOCK LLP, 353 SACRAMENTO STREET, SUITE 2200, SAN FRANCISCO, CA, 94111, US

(21) Appl. No.: 11/851,335

(22) Filed: Sep. 6, 2007

Related U.S. Application Data
(60) Provisional application No. 60/824,610, filed on Sep. 6, 2006.

(51) Int. Cl. A61N 7/00

(52) U.S. Cl. 601/2

(57) ABSTRACT

One embodiment of an ultrasound system for reducing the appearance of cellulite includes an ultrasound contact plate positioned within the cavity of a handpiece. Suction is used to draw tissue into the cavity, bringing the skin surface into contact with the ultrasound contact plate during ultrasound energy delivery. A motor mechanically vibrates the handpiece during ultrasound delivery, causing the contact plate to reciprocate relative to the underlying tissue undergoing ultrasound exposure.

This application claims the benefit of U.S. Provisional Application No. 60/824,610, filed Sep. 6, 2006.

### FIELD OF THE INVENTION

The present invention relates generally to dermatological treatment systems and methods using ultrasound energy, and more particularly for systems suitable for reducing the appearance of cellulite.

### BACKGROUND

Various non-invasive therapies are available for treating dermatological conditions using energy sources designed to cause heating within shallow regions of the skin. Such therapies generate heat using energy generated by lasers, flashlamps, or RF electrodes. These modalities have been described for treatment of skin laxity, wrinkles, cellulite, for removal of unwanted hair, and for other conditions.

Non-invasive ultrasound treatments are commonly used by physical therapists for treatment of pain conditions in muscles and surrounding soft tissue. To date, use of such treatments has not found commercial use as a dermatological therapy.

Cellulite is a well known skin condition commonly found on the thighs, hips and buttocks. Cellulite has the effect of producing a dimpled appearance on the surface of the skin.

In the human body, subcutaneous fat is contained beneath the skin by a network of tissue called the fibrous septae. When irregularities are present in the structure of the fibrous septae, lobules of fat can protrude into the dermis between anchor points of the septae, creating the appearance of cellulite.

There is a large demand for treatments that will reduce the appearance of cellulite for cosmetic purposes. Currently practiced interventions include lipsosuction and lipoplasty, massage, low level laser therapy, subscission surgery, mesotherapy, external topicals, creams and preparations such as “cosmeceuticals.” Lipsosuction and lipoplasty are effective surgical techniques through which subcutaneous fat is cut or suctioned from the body. These procedures may be supplemented by the application of ultrasonic energy to emulsify the fat prior to its removal. Although they effectively remove subcutaneous fat, the invasive nature of these procedures presents the inherent risks of surgery as well as excessive bleeding, trauma, and extended recovery times.

Non-invasive interventions for subcutaneous fat reduction are desirable but to date have yet to produce satisfactory results.

### BRIEF DESCRIPTION OF THE DRAWINGS

FIG. 1 is a perspective view of an embodiment of an ultrasound treatment system;

FIG. 2 is an enlarged perspective view of the handpiece of the system of FIG. 1;

FIG. 3 is a perspective view of the underside of the handpiece of FIG. 2;

FIG. 4 is an exploded perspective view of the operational components of the handpiece of FIG. 2;

FIG. 5 is a block diagram schematically representing the system of FIG. 1;

FIG. 6 illustrates an acoustic field generated by the transducers shown in FIG. 4;

FIG. 7 is an exploded perspective view of an alternative handpiece usable with the system of FIG. 1.

FIG. 8 is a perspective view of a second embodiment of an ultrasound treatment system;

FIG. 9 is a partial cross-section view of a handpiece of the embodiment of FIG. 8.

### DETAILED DESCRIPTION

The present application describes a system and method for non-invasive dermatological treatment using ultrasound. Systems of the type disclosed herein may be used to direct ultrasound energy into the skin, causing heat at depths selected to produce a desired effect, such as contraction of collagen for skin tightening, reducing the appearance of cellulite, or thermal damage or destruction of hair follicles for hair removal.

### First Embodiment

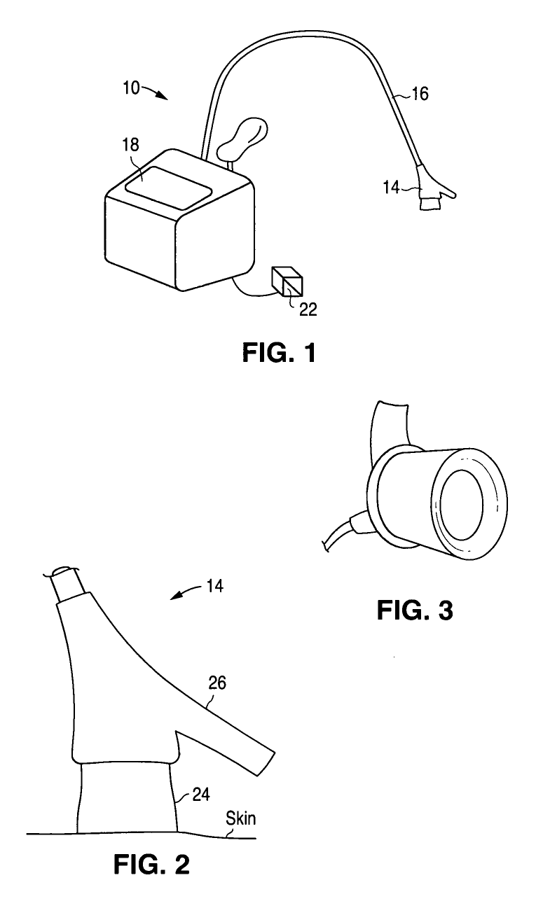

A first embodiment of an ultrasound treatment system 10 is illustrated in FIG. 1. System 10 uses therapeutic diathermy to heat target tissue. The first embodiment preferably, but optionally, combines diathermy with suction and vigorous massage of surrounding tissue using mechanical vibration. It has been found that the combination of these therapies can be an effective dermatological therapy, useful for improving the appearance of cellulite in the hips, thighs, and buttock areas of patients. Other therapeutic benefits include reduction of muscle pain and spasms and improved circulation.

System 10 includes a console 12 and a detachable handpiece 14 connected to the console with an umbilical cable 16. As will described in greater detail below, in a preferred mode of operation, the handpiece applies vacuum suction to a body area while delivering mechanical vibration and ultrasound energy to the tissue. Superficial tissue layers are preferably cooled before, during and/or after application of ultrasound energy.

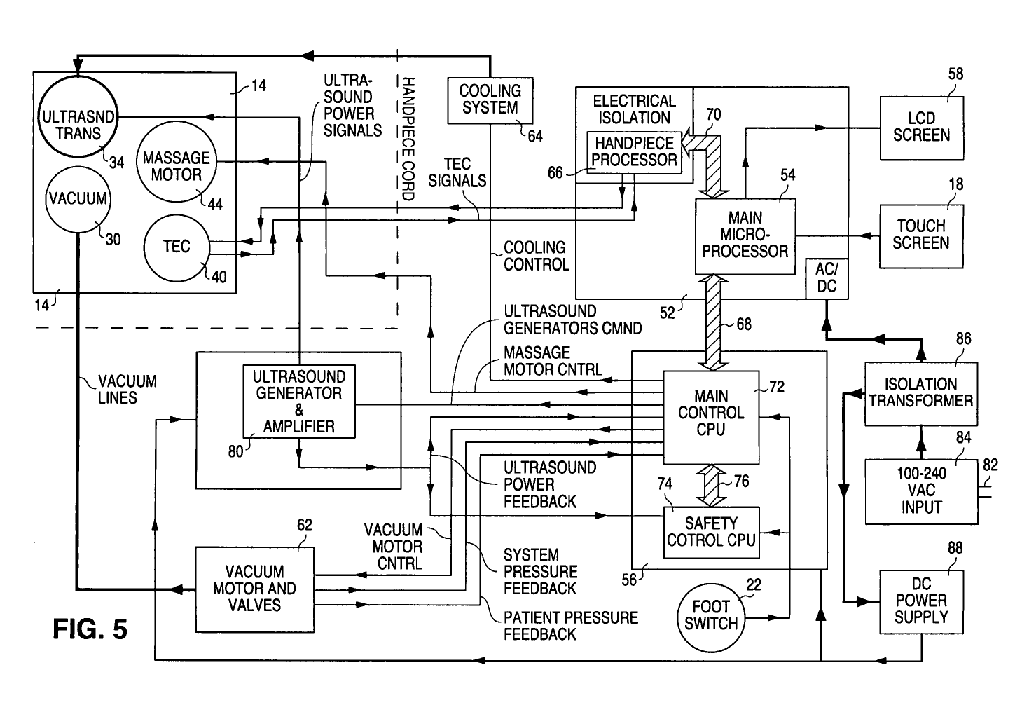

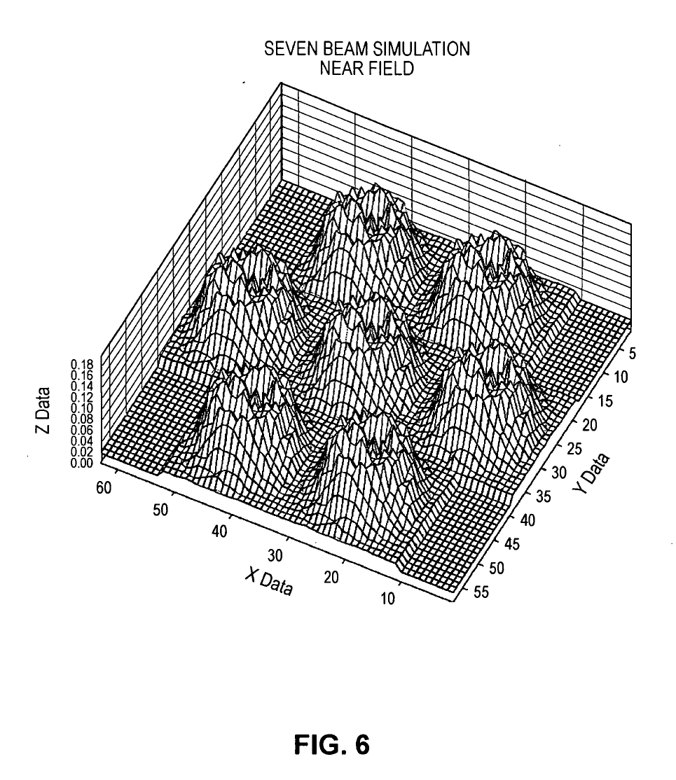

Console 12 includes a touch screen control panel 18 that allows a user to adjust treatment parameters and monitor the status of the system 10. A handpiece cradle 20 receives the handpiece when it is not in use. A footswitch 22 allows a user to activate a treatment sequence. Additional features of the console are discussed in connection with FIG. 5.

Referring to FIG. 2, handpiece 14 includes a fixation cup 24 positionable in contact with a patient's skin over the area to be treated. The fixation cup 24 is provided with dimensions appropriate for the dermatological application to be carried out. In one embodiment suitable for treatment of cellulite of the thighs and buttocks, a fixation cup 24 having a 4 inch diameter footprint is suitable. A handle 26 on the handpiece allows the user to move the cup 24 from one skin position to the next between treatment sequences. As shown in FIG. 3, a tissue contact plate 28 is mounted within the cup 24. Tissue contact plate 28 is formed of a material suitable for ultrasound transmission with sufficient thermal conductivity to allow superficial contact cooling of the skin. In one embodiment, tissue contact plate 28 is formed of aluminum having a gold coating on its tissue contacting surface Other suitable materials for contact plate 28 include, but are not limited to, bare aluminum, anodized aluminum, other metals such as copper, or thermally conductive crystalline solids such as sapphire or silicon nitride or boron nitride.

Vacuum ports 30 within the cup are coupled to a vacuum source (discussed in connection with FIG. 5), such that application of suction via the ports 30 will draw a patient's skin into contact with the tissue contact plate 28 and temporarily fix the cup 24 against the skin. Ports 30 may also be used to delivery a spray of liquid to skin prior to treatment, although the skin might instead be moistened using a separate spray bottle. Wetting the skin prior to treatment ensures adequate suction between the fixation cup 24 and the skin, and optimizes ultrasound coupling.

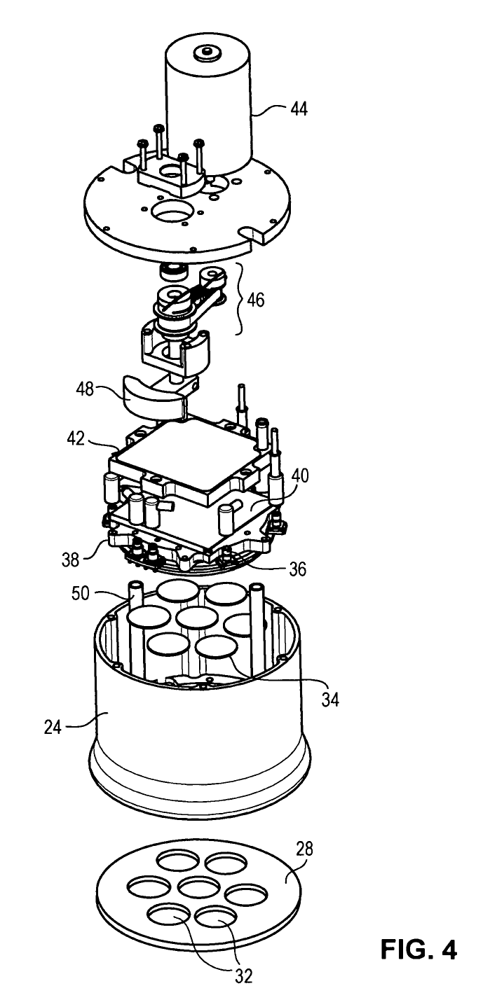

Operational components of the handpiece 14 are shown in the exploded view of FIG. 4. As shown, a plurality of recesses 32 is formed into the inwardly-facing surface of the contact plate 28. Piezoelectric transducers 34 seat within the recesses 32. The transducers may be arranged to produce collimated energy, or divergent or convergent energy patterns. Printed circuit boards 36 associated with each transducer 34 include the circuitry for driving the transducers.

The handpiece includes cooling features for (1) cooling the surface of the skin while the underlying tissue layers are heated by ultrasound energy; and (2) removing heat generated in the handpiece during operation. In particular, a heat spreader 38 formed of nickel plated copper or other thermally conductive material is positioned in contact with the inwardly facing surface of tissue contact plate 28. Heat spreader 38 is cooled by a thermo-electric cooler 40. A heat sink 42 positioned in contact with the back side of the thermo-electric cooler 40 draws away heat generated by the cooler 40. Heat sink 42 preferably includes micro-channels (not shown) through which cooling fluid circulates during use in a manner known to those skilled in the art. The system uses feedback from sensors in the handpiece to monitor the temperature of the ultrasound transducers and/or the temperature of the skin-cooling plate and control operation of the cooling features to ensure adequate surface cooling.

Various techniques can be used to mechanically manipulate the tissue. In the disclosed embodiment, the fixation cup 24 imparts mechanical vibrational energy to the tissue when the cup is engaged with the body tissue. In the illustrated embodiment, a motor 44 is coupled to a counterweight 48 by a belt drive system 46, such that rotation of the motor causes vibration of the fixation cup 24.

Vacuum lines 50 extend from the vacuum ports 30 (FIG. 3) through umbilical cable 16 (FIG. 1) to a vacuum motor. A filter trap (not shown) is positioned to collect debris and particles vacuumed into the vacuum lines 50 during the treatment cycle. The trap may be positioned within the handpiece, umbilical cable, or associated connectors.

The system architecture for the system 10 is illustrated in FIG. 5. The system includes the following main blocks: main processor board 52, main control board 56, LCD screen 58, touch screen 18, ultrasound generator board 60, vacuum system 62, hand piece 14, cooling system 64 and footswitch 22.

Main processor board 52 contains a main microprocessor 54 having an associated memory and input/output ports. Microprocessor 54 controls graphical user interface (GUI) features drawn on the system's LCD screen 58, receives user input (e.g. treatment parameters) from the touch screen 18 and communicates with the main control board 56 and an electrically isolated hand piece processor 66. The main microprocessor 54 and the main control board 56 communicate via a bidirectional serial link 68. Another bidirectional serial link 70 transmits communications between the hand piece processor and the main microprocessor 54.

The main control board 56 governs most of the system's hardware functionality. Main control board 56 includes a main control CPU 72, safety control CPU 74 and all necessary input/output ports. The main control CPU 72 receives commands from the main microprocessor 54 via serial link 68. Commands include exposure settings and limits, status requests and auxiliary commands.

Main control CPU 72 also maintains communication with safety control CPU 74 via a bidirectional serial link 76. Both of the control CPUs 72, 74 monitor the system footswitch 22 which is engaged by a user to activate treatment.

Main control CPU 72 controls the speed of the massage motor 44, ultrasound generators 80 on the ultrasound generator board 60, and the vacuum motor and valves 62. It also monitors the ultrasound power signal generated on the ultrasound generator board 60, as well as system and patient vacuum levels.

The safety control CPU 74, among other system tasks, monitors the ultrasound power signal generated on the ultrasound generator board 60, thus implementing a redundant power monitoring system.

The hand piece processor 66 receives commands from the main microprocessor 54 and executes temperature control tasks. This system controls the TEC (thermoelectric cooler) 40 located in the hand piece 14. Specifically, it receives temperature feedback signals needed for closed loop control.

Ultrasound generators and amplifiers 80 provide driver signals for the ultrasound transducers 34.

The vacuum ports 30 in the hand piece 12 receive suction from the vacuum system controller 62.

As discussed previously, the cooling system 64 contains a heat exchanger 42 (FIG. 4), a water reservoir and a pump. This system is designed to remove heat created in the hand piece during operation as well as enable skin temperature control facilitated by the TEC 40. It is controlled by main control CPU 72

System AC input comes from an AC wall plug 82 to input module 84.

Isolation transformer 86 feeds both the DC power supply 88 and on-board DC power supply located in the main processor board 52.

Operation of the system of FIG. 1 will next be described in the context of treatment of cellulite of the thighs and buttocks. First, using the system touch screen 18, the user selects the cycle duration (typically between 0 and 20 seconds) which corresponds to the duration of mechanical manipulation, and the massage intensity (on a scale of 1-10). The user additionally selects the ultrasound dosing time (typically between 3 and 8 seconds) and the heating dose, e.g. between 0-30 J/cm2.

Next, water or other liquid is applied to the skin overlaying the target area of cellulite. Referring to FIG. 2, the fixation cup 24 is then placed over the target area. The footswitch 22 is depressed. The vacuum system is activated, causing the cup 24 to engage the skin, and causing an area of skin to be drawn into the cup 24 and into contact with the tissue contact plate 28. In a preferred embodiment, vacuum pressure in the range of 5-20 atm, and most preferably approximately 10 atm is preferred.

While the tissue is engaged, the ultrasound transducers 34 are energized, preferably delivering continuous wave ultrasound energy to the tissue at a frequency in the range of 3-6 MHz, and most preferably approximately 5 MHz. The applied ultrasound has a preferred intensity in the range of 1-5 W/cm², with a preferred maximum temporal average intensity of approximately 5 W/cm² and a preferred maximum spatially averaged intensity of approximately 3 W/cm²over the entire contact surface. In the preferred embodiment the temporal average of the ultrasonic power is approximately 105 W±2-%.

The transducers may be energized simultaneously, or they may be sequentially energized according to a predetermined duty cycle.

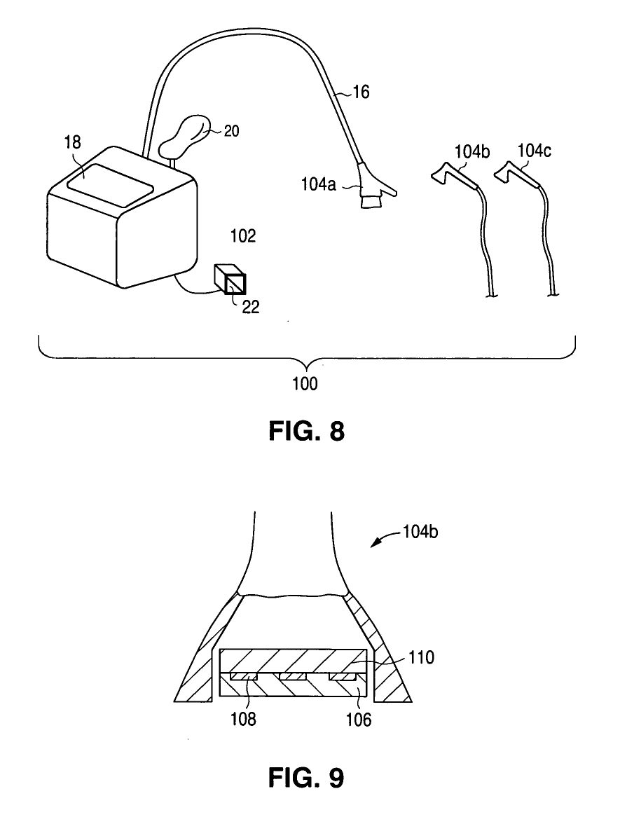

FIG. 8 shows a representative field map for the near ultrasound field produced from seven piezoelectric transducers arranged as in FIG. 4. The fields shown are representative of free propagation in a 25 C degassed water bath. The field amplitude units are arbitrary, while the lateral dimensions are given in millimeters. In the representative embodiment, individual transducers are spaced by a distance of 20-25 mm, measured from center-to-center of the individual transducers, however the array could have a variety of field patterns, depths and intensities. In alternate embodiments, certain ones of the transducers may be different from the others. For example, the outer ring of transducer elements might deliver energy at higher intensities than the inner one (or ones) which may be advantageous for producing a uniform heating profile if, for example, the center part of the target area does not require as much heating as the edges. For similar reasons, in some embodiments different ones of the elements may be operated at significantly different frequencies. For example, outer elements may be operated at a lower frequency than the inner elements to cause the outer elements to achieve a greater depth of energy penetration than the inner elements.

Mechanical manipulation also occurs during application of ultrasound energy. Mechanical manipulation and ultrasound delivery may commence simultaneously or at separate times. Rotation of the motor 44 causes the counterweight 48 to spin, resulting in eccentric lateral vibration of the cup 24. Although the ultrasound transducers are substantially fixed against the skin surface during treatment, vibration of the cup 24 causes lateral movement of the transducers relative to the subcutaneous tissue that is being treated. The vibration thus helps to “smooth out” the heating effects of the ultrasound in the tissue, giving more uniform heating and minimizing hot pockets within the tissue. In one embodiment, the counterweight produces a lateral vibration of approximately 30-70 Hz, preferably with enough force to produce redness/erythmea of the skin.

During ultrasound delivery, the tissue contact plate is cooled by the thermoelectric cooler, thereby maintaining the normal temperature of the skin and/or cooling the surface of the skin. In a preferred mode of treating cellulite, the ultrasound and cooling systems create a heating profile that produces a temperature rise in the subcutaneous of up to 10° C. while maintaining the epidermis at or below nominal body temperature, creating a reverse thermal gradient in the tissue that allows therapeutic temperatures to be achieved at depth with minimal collateral thermal damage to tissue surface. For other applications, such as reduction of skin laxity, the ultrasound and cooling parameters may be altered to alter the thermal profile to one that will give the appropriate therapeutic effects for shrinkage of collagen etc.

Throughout the treatment cycle, pressure sensors are used to generate feedback corresponding to the vacuum pressure of the system and the patient. If the pressure sensors detect that the cup 24 is not well sealed against the tissue, the treatment cycle will end and/or the console 12 will provide an auditory and/or visual alarm notifying the user that there may be inadequate contact between the handpiece and the skin. As an additional or alternative mechanism for evaluating the sufficiency of ultrasound coupling between the contact plate and the skin, the system can measure the electrical impedance or change in the voltage or current of the transducer amplifier. The measured impedance will increase if the transducer plate is not in contact with skin, for example.

Because bone tissue can be heated very rapidly by ultrasound energy, some embodiments might include features that notify the user when the handpiece is positioned less than a predetermined distance from an underlying bone. One example would be to look at the reflected ultrasound of the treatment pulse with a suitable transducer, another would analyze reflected ultrasound from additional low power ultrasound transducers to sense the presence of bone. These “diagnostic” transducers could operate at frequencies different from the treatment frequency to optimize resolution and/or allow filtering out of the treatment reflected ultrasound to increase signal of the diagnostic probe ultrasound signal. In either case, the system analyzes the reflected ultrasound to generate feedback corresponding to whether the handpiece is positioned within a certain distance from a patient's bone. A time of flight measurement type measurement might be made from a short duration or sharply switched ultrasound waveform. Alternatively, a simple amplitude or intensity measurement may suffice. In such embodiments, feedback that the handpiece is near an underlying bone can cause an auditory and/or visual alarm, and/or it may lockout the system against application of ultrasound until the handpiece is repositioned and/or the lock is overridden by the user.

At the end of the treatment cycle, ultrasound and mechanical energy transmission terminate, and suction is released. The user lifts the cup from the skin surfaces and repositions it at an adjacent tissue region. The process is repeated until the entire area to be treated has been exposed to treatment energy.

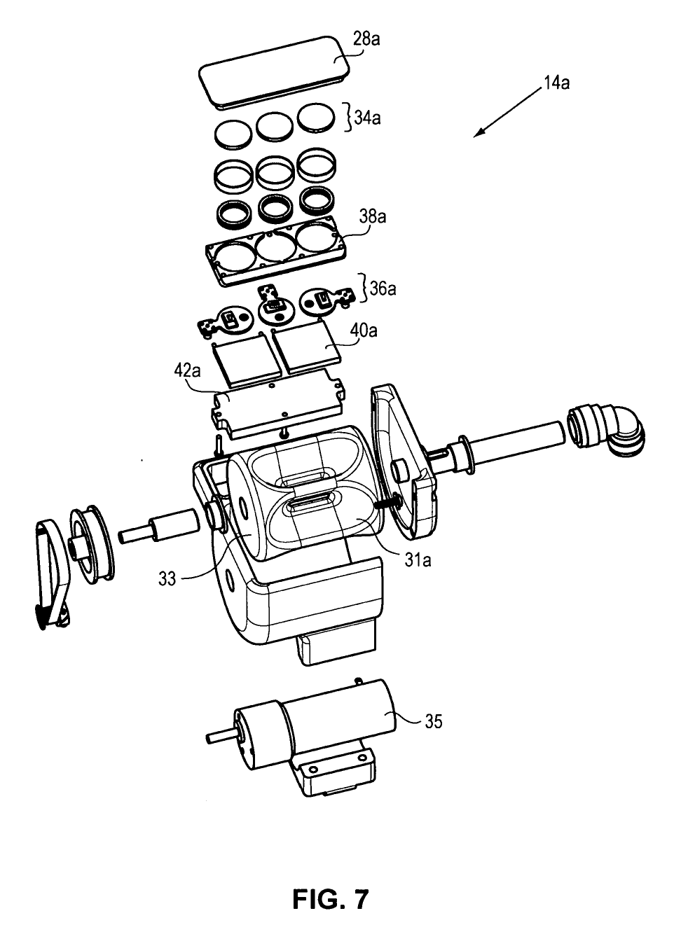

FIG. 7 shows an alternative handpiece 14a that may be used in the system of FIG. 1. The FIG. 7 handpiece differs from that of FIG. 4 in that it is configured to be moved across the surface of the skin during application of ultrasound energy. As shown, suction chambers 31a are positioned on a drum 33 rotated by a motor 35. Drum 33 rolls across the surface of the skin as the handpiece 14a is guided by a user, causing the suction chambers 31a to briefly engage and then detach from an area of skin. In the FIG. 7 embodiment, the contact plate 28a (through which energy from ultrasound transducers 34a passes into the skin) is positioned separate from the suction chambers, such that the contact plate 28a glides over the skin, trailing or leading the drum 33. Features such as a heat spreader 38a, printed circuit boards 38a, thermoelectric coolers 40a, and a heat sink are similar to those described in connection with FIG. 4 and will not be discussed in further detail.

### Second Embodiment

FIG. 8 shows a second embodiment of a dermatological ultrasound treatment system 100. The FIG. 8 system differs from the FIG. 1 system in that it is equipped to provide ultrasound therapy for a variety of purposes, such as skin tightening, hair removal, as well as cellulite reduction. FIG. 8 shows the system 100 as including a console 102 and a plurality of detachable handpieces 104a, 104b, 104c that may be selected for providing a desired treatment. For example, handpiece 104a may be a cellulite treatment handpiece of the type having the features described in connection with FIG. 4 or FIG. 7, or one that delivers ultrasound energy to the subcutaneous tissue without the use of mechanical manipulation and/or suction. Handpiece 104b may be a skin tightening handpiece useful for heating in shallower tissue regions to promote contraction of collagen; and handpiece 104c may be one configured for heating hair follicles for hair removal.

Although FIG. 8 shows a multi-application system having handpieces for different applications, dedicated systems configured for a particular procedure (e.g. skin tightening or hair removal or cellulite treatment may instead by used). Additionally, a single handpiece may be used to perform more than one type of treatment. For example, handpiece 104b may be operated in a skin tightening mode and in a separate hair removal mode.

Handpieces 104b and 104c are illustrated without the use of massage and suction functionality, although modifications may be made to provide those additional features.

An example of a handpiece 104b is illustrated schematically in FIG. 9. The handpiece includes a contact plate 106, one or more ultrasound transducers 108, and one or more cooling elements 110 that may be similar to the features discussed in connection with the FIG. 4 handpiece or others known in the art in connection with other modalities such as optical skin treatments. The associated printed boards, electrical conductors, and fluid lines are not shown in FIG. 9 for simplicity.

Handpiece 104b is operable to create a heated zone of tissue that is sufficiently shallow for collagen tightening. The operational frequency for the transducers 108, the amount of cooling performed using cooling features 110, and the amount of ultrasound power is selected to produce a thermal profile in the target tissue (which, for collagen heating is preferably a region where the heated zone is centered approximately 2-3 mm below the skin surface). In general, increasing the ultrasound frequency will give shallower penetration, but the depth of penetration is further influenced by the amount of heat drawn from the skin using the cooling system, and the amount of ultrasound power used. Once a target tissue volume and depth are selected, an operational frequency for the transducers is chosen that produces heating at the desired depth, and an intensity is selected to give the desired rate of heating (generally relatively slow for skin treatment). A cooling capacity is selected that keeps up with the evolution of heat to the surface, so that watts per square centimeter are “removed” at a particular temperature at which the skin surface is to be held. The combined effect of these parameters determines the shape of the thermal profile. In one example, the handpiece 104b may use transducers 108 operable at 10 Mhz at pulses of 1-10 seconds and an intensity of 1-3 W/cm2, in combination with cooling to remove 5-10 W/cm2 at the temperature (e.g. 20 C) at which skin temperature is to be clamped. Although parameters are given for collimated ultrasound transducers, the thermal profile can be altered to provide a focused or divergent ultrasound field.

Handpiece 104c may have features similar to those of handpiece 104b shown in FIG. 9. In an approach for selecting operating parameters for a handpiece such as handpiece 104c which relies on selectivity for heating, one first picks a target tissue structure (which for the purpose of this example is a hair follicle. Applied frequency and exposure time is selected to maximize energy selectivity and heating effect. The field may be shaped (e.g. using focusing) to locally increase the applied field at the target structure. Transducers operable to produce a divergent energy pattern may be used to give strong heating in the shallower tissue regions. Alternatively, the handpiece may produce multiple spaced apart fields of ultrasound energy focused to cause the greatest amount of heating at the hair follicles. Examples of operational parameters and handpieces for use in hair removal are shown and described in U.S. application Ser. No. _____, (Attorney Docket Number ALTU 2410), entitled ULTRASOUND SYSTEM AND METHOD FOR HAIR REMOVAL, filed Sep. 6, 2007, which is incorporated herein by reference.

Although the cooling element 110 is shown in FIG. 9 as behind the ultrasound transducers, other transducer positions may be used to optimize cooling. For example, the cooling element 110 may be a positioned adjacent to the contact plate 106 so that it directly contacts the skin. The position of the cooling element may be positioned so that as the contact plate 106 is moved across the surface of the skin, the cooling element 110 contacts a region of skin just before and/or after contact plate 106 has exposed that region to ultrasound energy. The cooling element might have an annular shape and be positioned surrounding the contact plate 106 such that it contacts tissue just exposed to ultrasound regardless of the direction in which the applicator is being moved. In other embodiments, the contact plate itself may be formed of an acoustically transmissive cooling material so that tissue is simultaneously exposed to cooling and ultrasound energy.

To use the handpieces 104b, 104c, an ultrasound coupling gel may be first applied to the tissue.

It should be recognized that a number of variations of the above-identified embodiments will be obvious to one of ordinary skill in the art in view of the foregoing description. For example, although a multi-modality system is disclosed, the various modalities may be combined in a variety of ways (including, but not limited to, ultrasound and cooling without suction and/or massage). Accordingly, the invention is not to be limited by those specific embodiments and methods of the present invention shown and described herein. Rather, the scope of the invention is to be defined by the following claims and their equivalents.

Any and all patents, patent applications and printed publications referred to above, including for purposes of priority, are incorporated by reference.

We claim:

1. A dermatological treatment device, including:
a handpiece;
an ultrasound applicator carried by the handpiece, the applicator having a contact surface positionable in contact with skin; and
a motor operable to mechanically vibrate the handpiece while the contact surface is in contact with skin.

2. The device of claim 1, wherein the motor is operable to mechanically vibrate the handpiece during delivery of ultrasound energy from the ultrasound applicator to the tissue.

3. The device of claim 1 wherein the handpiece includes a cavity, the contact surface positioned within the cavity, and wherein the device further includes a vacuum source coupled to the cavity, the vacuum source operable to draw tissue into the cavity when the opening is positioned to receive the tissue.

4. The device of claim 3 wherein the vacuum source is operable during delivery of ultrasound energy from the ultrasound applicator to the tissue.

5. The device of claim 1, wherein the ultrasound applicator further includes a cooling element positioned to cool the contact surface.

6. A dermatological treatment device, including:
a handpiece having a cavity;;
an ultrasound applicator carried by the handpiece, the applicator having a contact surface within the cavity and positionable in contact with skin; and
a vacuum source coupled to the opening, the vacuum source operable to draw tissue into the cavity when the cavity is positioned to receive the tissue.

7. The device of claim 6, wherein the ultrasound applicator further includes a cooling element positioned to cool the contact surface.

8. A dermatological treatment method, comprising the steps of:
using an ultrasound applicator positioned in contact with a skin surface, delivering ultrasound energy to tissue underlying the skin while applying suction to the skin.

9. The method according to claim 8, wherein the method reduces the appearance of cellulite.

10. The method according to claim 8 wherein applying suction to the skin includes drawing an area of tissue into a cavity in the ultrasound applicator.

11. The method of claim 10 wherein applying suction to the skin includes drawing the area of tissue into contact with an ultrasound contact plate disposed within the cavity.

12. The method of claim 11 further including the step of mechanically vibrating the ultrasound applicator during delivery of ultrasound energy.

13. The method of claim 12, wherein mechanically vibrating the ultrasound applicator causes lateral movement of the contact plate relative to the subcutaneous tissue that is being treated.

14. The method of claim 8, further including the step of cooling tissue in contact with the ultrasound applicator.
